(19) United States
(12) Patent Application Publication
(10) Pub. No.: US 2022/0304631 A1
(43) Pub. Date: Sep. 29, 2022

(54) MULTISENSOR PULMONARY ARTERY AND CAPILLARY PRESSURE MONITORING SYSTEM

(71) Applicants: THE REGENTS OF THE UNIVERSITY OF CALIFORNIA, Oakland, CA (US); SENSYDIA CORPORATION, Los Angeles, CA (US)

(72) Inventors: William J. Kaiser, Los Angeles, CA (US); Christopher Baek, Los Angeles, CA (US); Per Henrik Borgstrom, Charlestown, MA (US); Aman Mahajan, Los Angeles, CA (US); Kanav Saraf, Los Angeles, CA (US); Michael Wasko, Los Angeles, CA (US); Xu Zhang, Los Angeles, CA (US); Yi Zheng, Los Angeles, CA (US)

(73) Assignees: THE REGENTS OF THE UNIVERSITY OF CALIFORNIA, Oakland, CA (US); SENSYDIA CORPORATION, Los Angeles, CA (US)

(21) Appl. No.: 17/216,460

(22) Filed: Mar. 29, 2021

(51) Int. Cl. A61B 5/00; A61B 5/021; A61B 7/04; A61B 5/352; A61B 5/0205; G16H 50/30; G06N 3/08; G06N 3/04

(52) U.S. Cl. CPC A61B 5/7278; A61B 5/021; A61B 7/04; A61B 5/352; A61B 5/725; A61B 5/0205; A61B 5/7264; G16H 50/30; G06N 3/08; G06N 3/0445

(57) ABSTRACT

Systems and methods are provided for the non-invasive computation of Pulmonary Artery Pressure (and its components of mean, systolic and diastolic) (PAP) as well as Pulmonary Capillary Wedge Pressure (and its components of mean, A-Wave and V-Wave) (PCWP) using a wearable sensor device. Cardiac acoustic and electrocardiogram sensor signals are obtained and multiple temporal, amplitude-based, and spectral features are extracted from the signals. Extracted features from a subject are used as inputs for pre-trained classification, regression, or advanced machine learning models to provide an accurate computation of PAP and PCWP and their associated component values without surgery.

### CROSS-REFERENCE TO RELATED APPLICATIONS

Not Applicable

### STATEMENT REGARDING FEDERALLY SPONSORED RESEARCH OR DEVELOPMENT

Not Applicable

### NOTICE OF MATERIAL SUBJECT TO COPYRIGHT PROTECTION

A portion of the material in this patent document is subject to copyright protection under the copyright laws of the United States and of other countries. The owner of the copyright rights has no objection to the facsimile reproduction by anyone of the patent document or the patent disclosure, as it appears in the United States Patent and Trademark Office publicly available file or records, but otherwise reserves all copyright rights whatsoever. The copyright owner does not hereby waive any of its rights to have this patent document maintained in secrecy, including without limitation its rights pursuant to 37 C.F.R. § 1.14.

### BACKGROUND

### 1. Technical Field

This technology pertains generally to patient cardiac monitoring, and more particularly to Pulmonary Artery Pressure (PAP) and Pulmonary Capillary Wedge Pressure (PCWP) computations from electrocardiogram (ECG) and phonocardiogram (PCG) acoustic sensor signals.

### 2. Background

Congestive heart failure (CHF) is a debilitating disease of abnormal heart function that produces inadequate blood flow and a decline in intracardiac pressures that are necessary to adequately fulfill the metabolic needs of the tissues and organs of the body. Acute worsening of cardiac function is one of the most common causes for admission for hospital treatment and the leading contributor to high healthcare delivery costs.

A number of methods and techniques for evaluating and quantifying the CHF condition of a patient have been assessed, Such clinical evaluations are essential for guiding the treatment of patients with chronic heart failure. Optimum management of progressive CHF conditions in a patient requires constant monitoring and adjustments of therapy in response to any observed changes in the condition of the patient.

In many cases, CHF assessment and management requires monitoring of certain hemodynamic pressure-based parameters such as pulmonary artery pressure (PAP) and pulmonary capillary wedge pressure (PCWP) in addition to volume-based parameters such as stroke volume (SV) and ejection fraction (EF). The current gold-standard for measuring PAP and PCWP includes a point-in-time assessment with invasive right heart catheterization technology.

The pulmonary artery catheter that is used in this assessment has a pressure transducer with an inflatable member at the tip that is inserted into the pulmonary vasculature through the right heart. When the pressure transducer is positioned in the pulmonary artery, the Pulmonary Artery Pressure (PAP) waveform is obtained. When the pressure transducer is positioned in a branch of the pulmonary artery, the balloon member of the catheter tip is inflated that temporarily blocks blood flow in the artery and the steady-state pressure (i.e., PCWP) waveform is obtained. However, this method is invasive and the results may be inconsistent and imprecise due to the dependence of the measurement on catheter position, partial wedging, balloon overinflation, breathing cycle as well as variability in clinician interpretations.

Alternative methods involving implantable intracardiac pressure sensors exist. However, these methods are also highly invasive, costly, risky, and require the presence and support of expert technicians which defers the collection of valuable hemodynamic information until the CHF patent is critically ill or is hospitalized.

Accordingly, there is an urgent and unmet need for methods enabling non-invasive and accurate monitoring of critical hemodynamic pressure-based parameters characterizing heart function. Such methods can reduce the burden of heart disease through identification of patients at risk, provide an opportunity for early prevention and intervention of disease conditions, and enable better therapy adjustments in response to subject conditions.

### BRIEF SUMMARY

A Cardiac Performance System (CPS) and methods are provided that preferably incorporate sensors in a wearable computing device that can provide clinicians with critical assessment metrics for patient cardiac care. The system acquires signals from electrocardiogram (ECG) and phonocardiogram (PCG) acoustic sensors and extracts relevant features from the processed signals from many subjects for training and calibrating the system on these feature values to calculate values for PAP or PCWP and their components as well as implementing a static version of this trained system for independent operation thereafter.

The PAP and PCWP measurements are important diagnostic indicators of the cause and progression of CHF and the measurements facilitate the diagnosis, monitoring and treatment of disease advancement.

The preferred apparatus used in CPS is a wearable array of ECG and PCG sensors and a central processor that receives and processes the sensor signals to produce PAP and PCWP measurement outputs. In one embodiment the computer processor includes a communications link and the sensor signals are transmitted and processed in a second computer that displays the PAP and PCWP measurement outputs.

The measurement of (1) Pulmonary Artery Pressure (PAP, and its components of systolic, diastolic, and mean-PAP) corresponding to the blood pressure in the main pulmonary artery that carries deoxygenated blood from the right ventricle to the lungs, and (2) Pulmonary Capillary Wedge Pressure (PCWP, and its components of A-wave, V-wave, and mean-PCWP) corresponding to an indirect estimation of left atrial blood pressure are provided without the placement of a pulmonary catheter into the subject's body.

The PAP and PCWP values are dynamic measurements that show multiple variations within the same heartbeat, i.e., the PAP and PCWP are recorded as waveforms for each heartbeat. However, it is the specific values of peaks, valleys, and/or average pressures in these waveforms for each cardiac cycle, rather than relative trends, that carry diagnostic significance. These values are recorded as components of these waveforms: systolic-PAP (sPAP), or the pressure with which the right ventricle ejects blood into the pulmonary vasculature during systole), diastolic-PAP (dPAP), or the indirect measure of left ventricular end-diastolic pressure), and mean-PAP (mPAP), or the average PAP throughout one cardiac cycle) for PAP and PCWP A-wave (aPCWP), or the pressure of left atrial contraction), PCWP V-wave (vPCWP), or the pressure during passive filling of the left atrium against a closed mitral valve), and mean-PCWP (mPCWP), or the average PCWP throughout one cardiac cycle) for PCWP.

Elevated PAP and PCWP component values indicate that the heart is subjected to abnormal stress and provide data points required to differentiate between underlying pathologies such as pulmonary disease versus heart failure, or right heart failure versus left heart failure. Individuals with similar EF and SV values may show completely different PAP and PCWP component values, and these metrics are therefore independently useful for assessing heart function of patients with CHF. The PAP and PCWP values are additionally useful in informing which medications are most suitable for a heart failure patient and for determining whether or not a patient is responding to therapy.

The calibration and computation processes use temporal, amplitude-based, and spectral features as inputs for feature identification and extraction. Features used for the process are preferably the average values across all heartbeats or select high-quality heartbeats for a subject, in one embodiment. Average features are mapped to the desired output using one or several well-known classification or regression techniques such as neural networks, linear or nonlinear regression, Support Vector Machines, k-nearest neighbors, trees or random forests, and maximum likelihood. The features and classification techniques used for this purpose capture the intra-heartbeat variations in heart function, the anatomical variations in left and right heart function, and/or the variations in feature values across the breathing cycle.

Accordingly, one aspect of the present technology is to provide a Cardiac Performance System (CPS) that enables both point-in-time and/or continuous PAP and/or PCWP measurements with a wearable device providing clinicians with critical assessment metrics for patient care. In a preferred embodiment, CPS performs signal processing computations to characterize cardiac acoustic signals that are generated by cardiac hemodynamic flow, cardiac valve, and tissue motion. In another embodiment, signal processing is accompanied with one of several well-known classification, regression, or advanced machine learning methods to provide accurate computation of PAP (and its components systolic-PAP, diastolic-PAP, mean-PAP) and PCWP (and its components PCWP A-wave, PCWP V-wave, and mean-PCWP).

Another aspect of the technology is to provide a system and method for computing CPS-based pulmonary pressure values for a new patient in real-time without the need for an invasive right heart catheterization procedure.

Another aspect of the technology is to provide a wearable sensor system with continuous or periodic sensing, processing, calculating and displaying features that will accurately monitor PAP (and its components sPAP, dPAP, and mPAP) and PCWP (and its components PCWP A-Wave, PCWP V-Wave, and mPCWP) measurement for a patient.

Further aspects of the technology described herein will be brought out in the following portions of the specification, wherein the detailed description is for the purpose of fully disclosing preferred embodiments of the technology without placing limitations thereon.

### BRIEF DESCRIPTION OF THE SEVERAL VIEWS OF THE DRAWINGS

The technology described herein will be more fully understood by reference to the following drawings which are for illustrative purposes only:

FIG. 1 is a schematic flow diagram of an illustrative PAP (and its components systolic-PAP, diastolic-PAP, mean-PAP) and PCWP (and its components PCWP A-wave, PCWP V-wave, and mean-PCWP) computation process from sensor signal data according to one embodiment of the technology.

FIG. 2A through FIG. 2E show images of an illustrative PCG pre-processing and R wave detection scheme for generating a high-quality clean ECG signal.

FIG. 3A through FIG. 3D are plots of an illustrative PCG signal noise suppression scheme in accordance with the one embodiment of the present technology.

FIG. 4A through FIG. 4C show plots of the PCG signal segment, low-frequency envelope and autocorrelation of consecutive cardiac cycles, respectively.

FIG. 5A through FIG. 5C show a cross-correlation method of estimating S1 locations.

FIG. 6A through FIG. 6C show a method for autocorrelation of the high-frequency envelope segment for systolic interval and subsequent S2 estimation.

FIG. 7A is an illustrative ECG waveform for two consecutive cardiac cycles as acquired in a right heart catheterization procedure.

FIG. 7B is an illustrative PAP waveform for the same consecutive cardiac cycles acquired in a right heart catheterization procedure of FIG. 7A.

FIG. 8A is an illustrative ECG waveform for two consecutive cardiac cycles as acquired in a right heart catheterization procedure.

FIG. 8B is an illustrative PCWP waveform for the same consecutive cardiac cycles acquired in a right heart catheterization procedure of FIG. 8A.

FIG. 9A is a relative PCG amplitude graph of a heartbeat over time.

FIG. 9B is an example of a formant frequency feature and its variations across different segments of the same heartbeat of FIG. 9A.

FIG. 10A is a graph of relative PCG amplitude over time for a heartbeat as recorded at the aortic acoustic sensor locations.

FIG. 10B is a graph illustrating an example of a formant amplitude feature for the same heartbeat recorded at the aortic acoustic sensor locations as shown in FIG. 10A.

FIG. 10C is a graph of relative PCG amplitude over time for the same heartbeat as shown in FIG. 10A but as recorded at the pulmonic acoustic sensor locations instead of the aortic acoustic sensor location.

FIG. 10D is a graph illustrating an example of a formant amplitude feature for the same heartbeat recorded at the pulmonic acoustic sensor locations as shown in FIG. 10C.

FIG. 11A is a plot of sPAP computations for an illustrative set of subjects.

FIG. 11B is a plot of dPAP computations for an illustrative set of subjects.

FIG. 11C is a plot of mPAP computations for an illustrative set of subjects.

FIG. 12A is a plot of PCWP A-wave computations for an illustrative set of subjects.

FIG. 12B is a plot of PCWP V-wave computations for an illustrative set of subjects.

FIG. 12C is a plot of mPCWP computations for an illustrative set of subjects.

FIG. 13 shows a schematic diagram of CPS monitor for measuring pulmonary pressure measurements with processor and sensors according to one embodiment of the present technology.

FIG. 14A shows an image of representative CPS acoustic sensor locations based on typical auscultatory sites used with a standard stethoscope system.

FIG. 14B shows an image of representative ECG sensor electrodes locations applied at conventional RA (right arm), LA (left arm), and LL (left leg) monitoring sites.

FIG. 15 illustrates a schematic diagram of an embodiment of the CPS sensor support without acoustic sensors.

FIG. 16 illustrates a schematic diagram of the CPS sensor support with multiple acoustic sensors to form an CPS sensor application system positioned around the abdomen of the patient.

FIG. 17 is a side view of an CPS acoustic sensor in accordance with the present technology.

### DETAILED DESCRIPTION

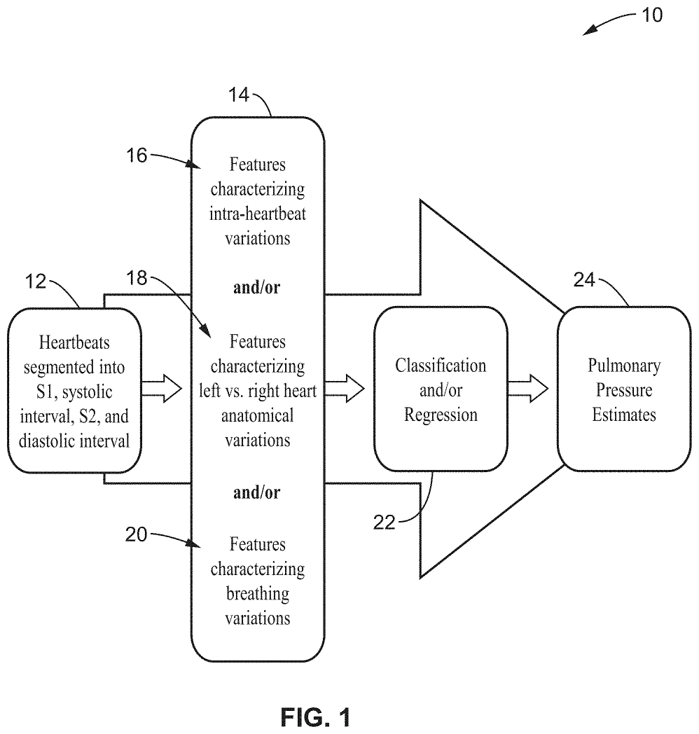

Referring more specifically to the drawings, for illustrative purposes, systems and methods for computing pulmonary artery pressure (PAP) and pulmonary capillary wedge pressure (PCWP) from acquired ECG and PCG acoustic signals of a patient are generally shown. Several embodiments of the technology are described generally in FIG. 1 to FIG. 17 to illustrate the characteristics and functionality of the devices, systems and methods. It will be appreciated that the methods may vary as to the specific steps and sequence and the systems and apparatus may vary as to structural details without departing from the basic concepts as disclosed herein. The method steps are merely exemplary of the order that these steps may occur. The steps may occur in any order that is desired, such that it still performs the goals of the claimed technology.

One important diagnostic indicator of the CHF condition is the measurement of Pulmonary Artery Pressure (and its components of mean, systolic and diastolic) (PAP) as well as Pulmonary Capillary Wedge Pressure (and its components of mean, A-Wave and V-Wave) (PCWP). Elevated values of these pressures indicate the presence of CHF condition.

The Cardiac Performance System (CPS) illustrated herein enables both point-in-time and/or continuous measurements of PAP (and its components sPAP, dPAP, and mPAP) and PCWP (and its components PCWP A-Wave, PCWP V-Wave, and mPCWP) with a wearable device providing clinicians with critical assessment metrics for patient cardiac care. Specifically, in one embodiment, CPS utilizes compact, wearable acoustic sensor devices and ECG sensor electrodes in a convenient patient belt or adhesive attachment application system. CPS performs signal processing to characterize heart sound signals that are generated by cardiac hemodynamic flow, cardiac valve, and tissue motion. Signal processing is accompanied with one of several well-known classification, regression, or advanced machine learning methods to provide accurate computation of PAP and PCWP and their associated component values.

The CPS system is non-invasive and supports clinical patient care via convenient point-in-time and/or continuous monitoring, which ensures patient safety and provides benefits to patients and clinicians as well as hospital facilities that can advance fundamental care. CPS is also advantageous for outpatient treatment by providing cardiac function monitoring to patients who would otherwise not receive an assessment. Finally, CPS further provides the ability for residential monitoring of heart function and remote diagnostic capabilities enabling early intervention and advanced perioperative care delivery.

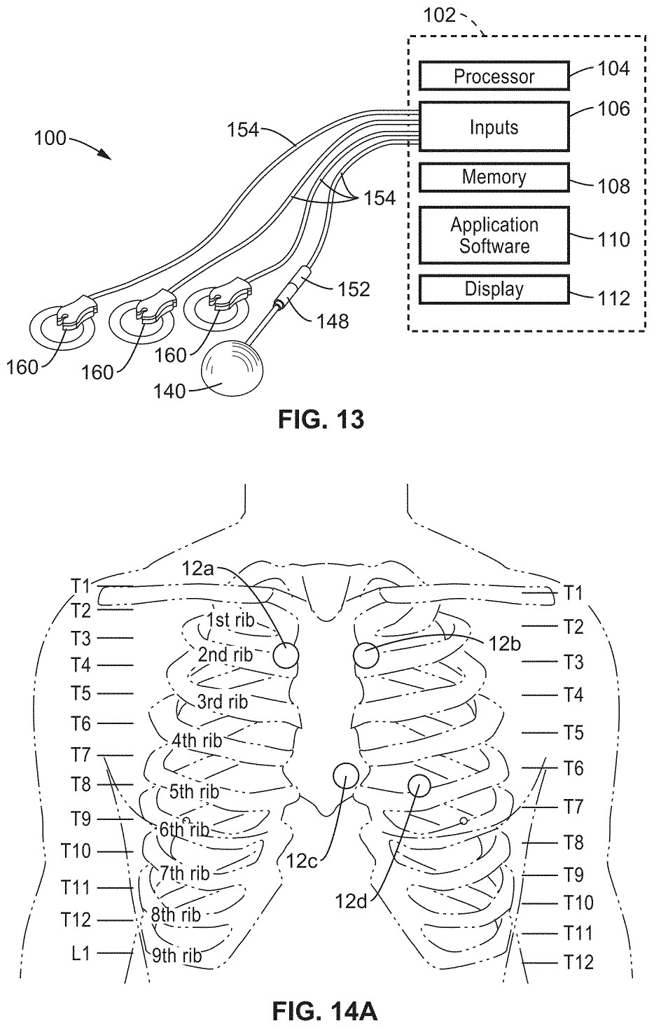

Turning now to FIG. 1, an embodiment of the method 10 for the computation of PAP (and its components) and PCWP (and its components) is shown schematically. The methods shown in FIG. 1 are preferably implemented as instructions in machine-readable code within one or more modules of application programing in a computation device that is part of the CPS system as illustrated in FIG. 13 to FIG. 17 or processed on an external processing device and displayed.

Overall, the calibration and computation processes use temporal, amplitude-based, and spectral features as inputs and produce PAP or PCWP values as outputs. Computations of PAP and PCWP are based on analysis of S1 (first heart sound), systolic interval, S2 (second heart sound), and diastolic interval characteristics, their timing relative to the QRS event in the ECG signal, differences in them over time, differences in them as reflected across acoustic signals acquired at the multiple acoustic sensor locations, and variations in them as reflected across the breathing cycle.

As seen in FIG. 1, the PCG signal analysis 10 generally comprises three main stages: pre-processing and segmentation; feature extraction, and classification/regression. At block 12 of the functional block diagram, sensor signal data from PCG and ECG sensors is acquired from a subject. The PCG and ECG sensor signals may optionally be processed for noise reduction and R-wave detection before segmentation. The acquired PCG and ECG sensor signals are continuously processed and segmented into S1, systolic interval, S2 and diastolic interval segments as inputs at block 12 of FIG. 1.

In one embodiment, the acquired PCG signal is processed for noise suppression and the ECG signal is used to segment the PCG signal (an ECG-gated segmentation method) to provide inputs at block 12. The ECG signals are measured using traditional ECG electrodes and used to enable timing and proper identification of phonocardiogram (PCG) acoustic signatures as belonging to S1, S2, or another part of the cardiac cycle. In each cardiac cycle, electrical depolarization of the ventricles causes a displacement in voltage observed in the ECG signal, known as the R wave. The R wave is usually the most prominent feature in the ECG signal. If the R wave can be accurately identified within each cardiac cycle, the signal can then be decomposed into individual cardiac cycles to segment the ECG signal. If the ECG and PCG are acquired synchronously, this same decomposition can be applied to the PCG. Thus, a primary objective of ECG signal processing when implemented in the methods of this embodiment is robust R wave detection.

R wave detection may be complicated by a number of factors. First, the amplitude and morphology of the R wave can vary widely due to variations in ECG electrode placement or the presence of certain cardiac conditions. These causes also contribute to variability in the amplitude of the T wave. The T wave of the ECG reflects the electrical repolarization of the ventricles in the cardiac cycle. In some scenarios, this may result in R and T waves of similar amplitude. This creates difficulty when attempting to identify R waves based solely on amplitude criteria.

In addition, several sources of noise can corrupt the acquired ECG signal, including: 1) power line interference; 2) electrode contact noise; 3) motion artifacts; 4) muscle contraction, and 5) baseline drift and amplitude modulation with respiration. Power line interference often includes 60 Hz noise that can be up to 50 percent of peak-to-peak ECG amplitude. Baseline drift and amplitude modulation may often result from respiration by the subject, creating large periodic variations in the ECG baseline. Electrode contact noise is caused by degradation of coupling between the electrode and the skin. The level of noise induced is dependent upon the severity of the degradation. If there is complete loss of contact between the electrode and skin, the system is effectively disconnected, resulting in large artifacts in the ECG signal. If coupling is reduced but there is still some degree of contact between electrode and skin, a lower amplitude noise is introduced, which may persist as long as the coupling is suboptimal. Coupling issues can also be intensified by subject motion and muscle contraction, which can further affect the contact surface area between electrode and skin.

To mitigate these effects, advanced pre-processing techniques may be used and implemented within application programming. Suitable pre-processing techniques include: (a) Band-pass filtering the acquired ECG signal; (b) Multiplication of the filtered signal by its derivative; (c) Envelope computation; (d) Identification of R waves in the computed envelope; (e) Identification of corresponding peaks in the filtered signal; and (f) Determination of R wave onset in the filtered signal.

Band pass-filtering may be used to minimize the effects of baseline drift, powerline interference, and other noise sources while maintaining underlying ECG signals. A band pass filter can be defined by its lower and upper cut-off frequencies, and the region between these two frequencies is known as the pass band. While optimal cut-off frequencies may vary based on hardware, an example embodiment may have a passband between 1 Hz and 30 Hz. There exist a large number of well-defined filter design tools both for Infinite Impulse Response (IIR) and Finite Impulse Response (FIR) filters which allow for the design of bandpass filters based on desired specifications for block-band rejection, passband attenuation, filter order, and other performance specifications. In CPS, the application of a bandpass filter can significantly improve the signal to noise ratio, and subsequent preprocessing may be performed on the filtered signal, ƒ(t).

In typical ECG signals, the R wave may be characterized by a large amplitude, and selection of R wave candidates based purely on amplitude can be effective. However, in some cases, T waves can become as prominent as R waves making it difficult to differentiate between waves. However, since R waves have a higher frequency content relative to typical T waves, the effect of elevated T waves can be differentiated. By computing the derivative of the signal ƒ(t), an operation that amplifies high frequency content, a signal with exaggerated R wave amplitude is generated. Subsequent multiplication of ƒ(t) with its derivative yields a new signal, g(t), that greatly emphasizes R waves relative to the sometimes-problematic T wave.

The envelope of the resulting signal, g(t), is computed using the Hilbert transform, and this envelope is subsequently low-pass filtered with a cutoff frequency of 8 Hz to further amplify the R wave, and the resulting envelope is normalized by dividing by its 98^{th} percentile value in this embodiment. It should be noted that this approach is used rather than division by the maximum value to reduce the effects of spurious outliers in the envelope.

Peak detection of the resulting signal may leverage known peak-detection algorithms with minimal peak height set to 50% of the maximum envelope height, for example. A number of conditions can be imposed to eliminate peaks not likely to be associated with R waves. For example, excessive amplitude or an excessive number of peaks in rapid succession can be used to guide removal of false peaks prior to subsequent processing. Once the R wave peak locations have been identified in the envelope, R wave onset is determined as the last value above a certain threshold. An example threshold here might be 50% of the envelope peak.

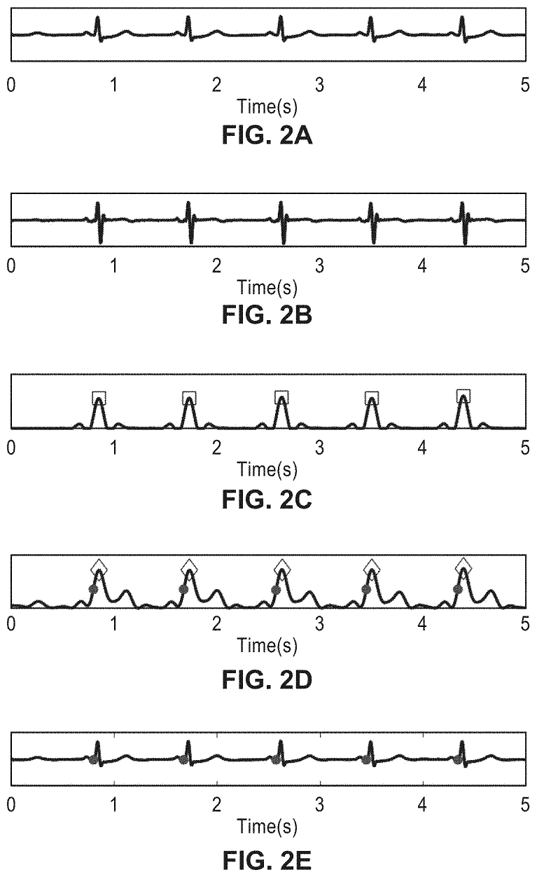

An example of PCG preprocessing and R wave detection at block 12 of FIG. 1 is shown in FIG. 2A through FIG. 2E that produces a high-quality and clean ECG signal. FIG. 2A is a continuous plot showing the raw ECG signal. FIG. 2B is a plot of the derivative of the filtered ECG signal. FIG. 2C illustrates the envelope of function resulting from multiplying signal by its derivative, with detected peaks marked by squares. FIG. 2D shows the envelope of filtered signal, with detected peaks marked by diamonds, and R wave onset marked by solid circles. FIG. 2E is a plot of the filtered ECG signal, with the R wave onset marked by solid circles. This ECG data may be used to segment the preferably synchronously acquired PCG data as described below.

The PCG sensor signals may also be processed prior to segmentation at block 12 of FIG. 1 to optimize the inputs. The PCG signal is often susceptible to noise from a wide variety of sources such as involuntary subject activity, voluntary subject activity, external contact with the PCG sensor, and environmental noise.

Involuntary subject activity includes involuntary physiological activity of the subject, such as respiratory and digestive sounds. Another common noise source in this group is the microscopic movement of tissue beneath the sensor, even with a seemingly motionless subject. This motion causes persistent fluctuations in the PCG signal that are usually of relatively low amplitude. If the cardiac signal strength is low, however, this noise can mask underlying cardiac events.

Voluntary subject activity includes activity such as speech and subject motion. These noise sources will generally create large disturbances in the PCG signal. Similarly, external contact with the sensor housing by another object such as clothing or a hand can also produce large artifacts in the signal.

Environmental noise includes all external sources of noise not involving the subject or the sensor. This may include non-subject speech, background music/television, and hospital equipment noise. With proper coupling of the sensor to the tissue, such noise factors typically have minimal effect on PCG signal quality, except for in extreme cases.

In one embodiment, PCG signal preprocessing preferably comprises band-pass filtering followed by Short-Time Spectral Amplitude log Minimum Mean Square Error (STSA-log-MMSE) noise suppression. Band-pass filtering may be performed with cut-off frequencies of 25 Hz and 100 Hz, which has been found to preserve PCG signals while reducing the amplitude of out-of-band noise sources.

In one embodiment, a model of signal noise is generated, and short time segments of data are considered. A probability of the presence of acoustic activity other than noise is computed for each time segment, and a gain is computed as a function of this probability. Gain is low for low probabilities and approaches unity for high probabilities, thereby reducing the amplitude of purely noise-segments of audio. It should be noted that these models and corresponding gains are considered in the frequency domain. Conversions to frequency domain are performed using the Fast Fourier Transform (FFT), and conversions back to the temporal domain are performed using the Inverse Fast Fourier Transform (IFFT).

For PCG analysis, adaptations to the STSA-log-MMSE algorithm can be made. Whereas typical STSA-log-MMSE applications generally require a recording of known noise-only data, pre-existing knowledge of the timing of the cardiac cycle based on ECG segmentation can be leveraged to determine regions of acoustic inactivity. For example, it is known that within each cardiac cycle there will be regions that contain no cardiac sounds. Even if all cardiac sounds, including murmurs, are present, there are regions without such sounds. Thus, the regions of each cardiac cycle with RMS energy in the 25th percentile are likely to be characterized by a minimal cardiac acoustic signature. This allows for online generation of noise models and for adaptive updating of such models.

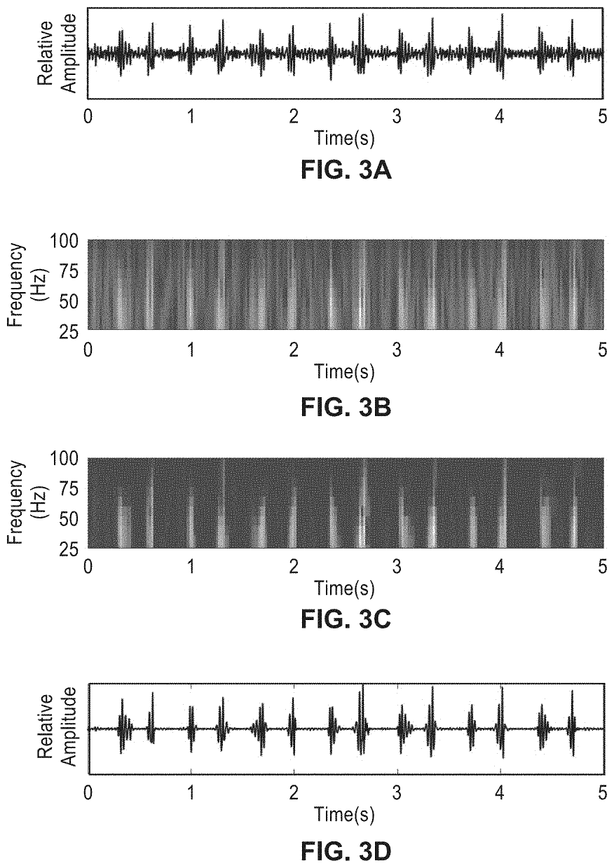

FIG. 3A through FIG. 3D illustrate an example of PCG signal noise suppression in accordance with the present description. FIG. 3A is a plot showing an original band-pass filtered PCG signal. FIG. 3B shows a spectrogram of original signal. FIG. 3C shows a spectrogram of the de-noised or noise-suppressed signal, which demonstrates a significant reduction in noise. FIG. 3D shows the final de-noised PCG signal, also demonstrating a significant reduction in noise.

In the segmentation stage of the inputs at block 12, cardiac acoustic events are preferably detected and labeled. These events may include the S1, S2, S3, and S4 sounds, as well as murmurs. In one embodiment, the CPS system and methods additionally leverage the systolic and diastolic intervals between S1 and S2 heart sounds. While the S1 and S2 heart sounds carry information about valve motion, systolic and diastolic intervals carry information about contraction and relaxation of heart muscles, tissue motion, and blood flow.

Segmentation at block 12 is preferably accomplished with one of two methods: 1) PCG-gated segmentation or 2) ECG-gated segmentation. In the case of PCG-gated segmentation, the PCG signal is segmented by sole examination of the PCG signal itself, without any complementary information from a synchronous ECG signal. Generally, in this approach, there is first a detection stage, where an event detection method is applied to locate heart sounds. Here, for example, signal processing methods are applied to emphasize regions of cardiac activity in the signal. Then, a decision method is applied to identify heart sounds based on certain predefined criteria.

Next, in the labeling stage, the detected sounds are labeled as one of the types described above. Typically, this stage focuses mainly on the S1 and S2 sounds, the interval duration between successive events, as well as characteristics of the events themselves, to identify which group a certain event belongs to for labeling. The interval between S1 and S2 of the same cardiac cycle is the systolic interval, and the interval between S2 of one cardiac cycle and S1 of the next cardiac cycle is the diastolic interval.

However, in PCG-gated segmentation, it is unknown a priori where the breakpoints of each cardiac cycle lie. Thus, when presented with two consecutive events, it can be challenging to determine whether they correspond to the S1 and S2 events of the nth cardiac cycle, or the S2 event of the nth cycle, and the S1 event of the (n+1)th cycle.

Finally, in the decomposition stage, the PCG signal is decomposed into individual cardiac cycles, with the corresponding events and intervals between events occurring during each cycle attributed to it. This allows for analysis of each cardiac cycle individually.

In contrast, with ECG-gated segmentation at block 12, an ECG-gated framework is implemented that analyzes the ECG signal and the R wave onset to enable timing of PCG signal segmentation. This method utilizes short-time periodicity of the ECG and PCG signals, a property that exists even in cases of abnormal heart rate.

To ensure periodicity, the PCG signal is analyzed in segments containing two consecutive cardiac cycles. Assuming the systolic intervals of consecutive cardiac cycles are consistent (which has been found to be the case, even in conditions of arrhythmia), performing correlation method analysis on such a segment allows for the accurate detection and labeling of S1 and S2 sounds.

The first step in PCG segmentation is the generation of PCG envelopes from the processed, noise-reduced signal described above. Envelopes may be generated using the Hilbert transform or by computing the absolute value of the signal and passing it through a low-pass filter. A number of different corner frequencies may be considered, and several envelopes may be generated and used for subsequent processing. Finally, the signal may be adjusted by raising it to some power less than 1 and applying a transform which tends to normalize the heights of peaks in the envelope such that all peaks are weighted approximately the same.

The envelopes are subsequently analyzed in segments containing two heartbeats that is a preliminary segmentation that is enabled by analysis of the high-quality ECG signals generated previously. Each heartbeat is processed as the second event in one window and as the first event in the next window. As such, each cardiac cycle is analyzed twice, thereby increasing the likelihood of proper detection of that beat.

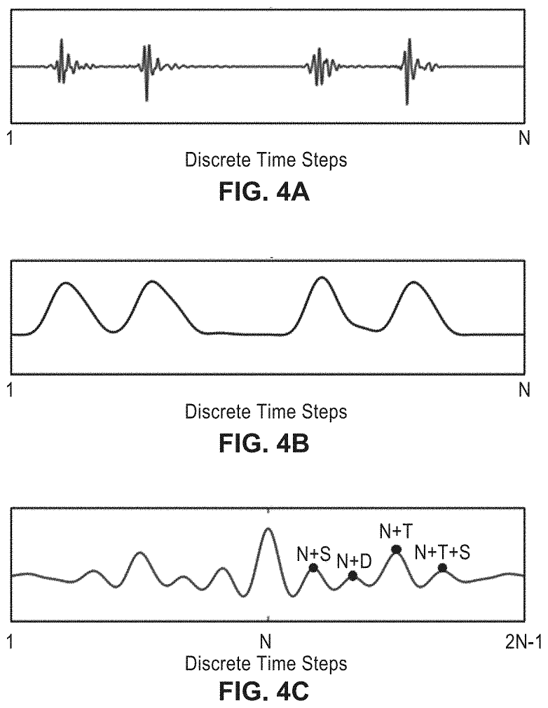

In one embodiment, an autocorrelation function is applied to each two-beat envelope. This operator is commonly used to detect periodicity in signals, and this property is useful in the PCG signal analysis. This process is highlighted in FIG. 4A through FIG. 4C. FIG. 4A shows a plot of the PCG signal segment of consecutive cardiac cycles. FIG. 4B shows a plot of the low-frequency envelope of corresponding segment. FIG. 4C shows a plot of the autocorrelation of low-frequency envelope. In FIG. 4C, several of the peaks are labeled by the corresponding intervals represented. It should be noted that there is a difference in scaling in the x axis between the plots shown in FIG. 4A through FIG. 4C.

The envelope shown in FIG. 4B is subjected to the autocorrelation operator, resulting in the symmetric signal, a(t), as shown in FIG. 4C. The a(t) shows a central peak, corresponding to the dot product of the envelope with itself with zero-time shift. There is also a second primary peak that is shifted by one period, T, relative to this central peak. This corresponds to the dot product of the envelope with an envelope shifted by T, such that the peaks associated with one heartbeat are aligned with those of the subsequent beat, thereby resulting in positive interference. Also evident in FIG. 4C are smaller peaks shifted by the systolic and diastolic periods (S and D), which are caused by overlap of S1 peaks with S2 peaks.

The autocorrelation described above enables computation of a valuable quality metric. For high quality PCG recordings, the peak at N+T is sharp and prominent. This prominence is quantified as the difference in its height relative to the lowest points surrounding it. This signal quality index is used to quantify signal quality, which is of critical importance in guiding subsequent algorithms. For example, if one sensor is characterized by low quality relative to others, its role in a classifier may be devalued or de-weighted relative to that of others. Alternatively, this feature can be used to alert system operators of insufficient signal quality, indicative of poor sensor placement.

Now that the cardiac period Tis determined, the next step is to determine the location of individual cardiac events within the cardiac cycle. To locate S1 events, a comb function may be generated whose value is zero at all locations except at integer multiples of the period. Convolution of this function with the PCG envelope yields a series of peaks as the delta functions in the comb pass through peaks in the envelope. When these deltas align with S1 events, a large peak is generated, and the offset of this peak is equal to the offset of the S1 events in the PCG signal. This yields a search interval in the original PCG signal within which the S1 event is known to occur.

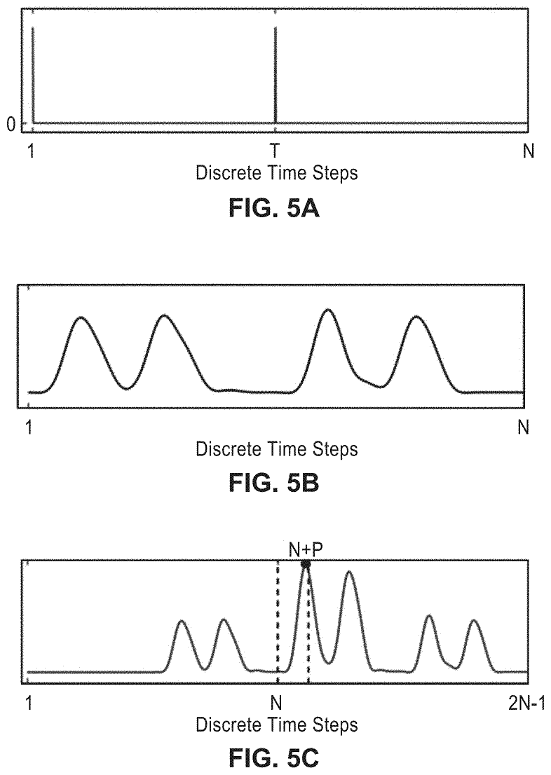

This process is demonstrated in FIG. 5A through FIG. 5C, which illustrate a cross-correlation method of estimating S1 locations. FIG. 5A shows a plot of function ƒ(n). FIG. 5B shows a plot of the low-frequency envelope of the PCG signal segment and FIG. 5C shows a plot of the cross-correlation of ƒ(n) with the low-frequency envelope. In FIG. 5C, the Si peak search interval is marked with dashed lines and the lag, P corresponding to the peak in this interval is the location estimate for S1 in the low-frequency envelope segment.

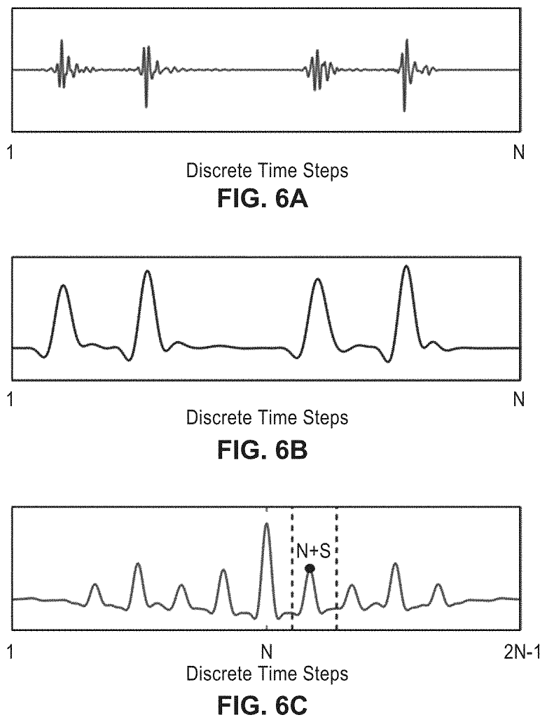

With the S1 peak located, the remaining task is to determine the S2 location. To this end, the autocorrelation, a(t), of the PCG envelope is revisited. As described above, a(t) contains secondary peaks associated with the systolic and diastolic time intervals (S and D). The systolic interval is given by the location of the first peak after the central peak as shown in FIG. 6A through FIG. 6C. Thus, the search region for S2 events is confined to the area around this peak. Because S2 events are not always evident in PCG signals, these peaks may not be discernible, and a search for a peak in this vicinity may yield peaks in regions where the S2 event is known not to occur. Thus, the search is limited to the region bounded by N+0.2T at one end and N+0.55T on the other. Peaks outside of this interval are not considered. This process is demonstrated in FIG. 6A through FIG. 6C, which illustrate autocorrelation of the high-frequency envelope segment for the systolic interval estimation. FIG. 6A shows a plot of the PCG signal segment. FIG. 6B shows a plot of the high-frequency envelope. FIG. 6C shows the resulting autocorrelation of the high-frequency envelope. In FIG. 6C, the dashed lines represent the boundaries N+0.2T<n<N+0.55T.

As a final step in PCG signal segmentation, false event removal methods may be applied. This may leverage timing and duration properties, as well as other known signal characteristics. For example, the time interval between onset of the R wave and onset of the S1 sounds is typically very consistent, a property than can be leveraged to remove detected S1 peaks that occur significantly before or after the expected time.

Additionally, cardiac events may be characterized by durations of approximately 20 ms to approximately 250 ms. If a detected peak has a duration outside of this range, it is likely that it is an artifact of noise and can be removed from consideration. Additional false event removal methods may involve the identification of systolic and diastolic interval signal excursions greater than 50% of S1 or S2 peak height, for example. Advanced quality assurance methods may employ several well-known classification or regression techniques including neural networks, linear or nonlinear regression, Support Vector Machines, k-nearest neighbors, trees or random forests, and maximum likelihood on a heartbeat-by-heartbeat basis to determine if a heartbeat is similar in appearance and characteristics to previously seen high-quality heartbeats.

Once the inputs are obtained at block 12 of FIG. 1, pertinent features are identified and extracted at block 14. In a preferred embodiment, the systems and methods 10 may be optimized during system training and calibration to utilize extensive prior studies performed on healthy and afflicted individuals with features shown to correlate with PAP (and its components systolic-PAP, diastolic-PAP, mean-PAP), and PCWP (and its components PCWP A-wave, PCWP V-wave, and mean-PCWP).

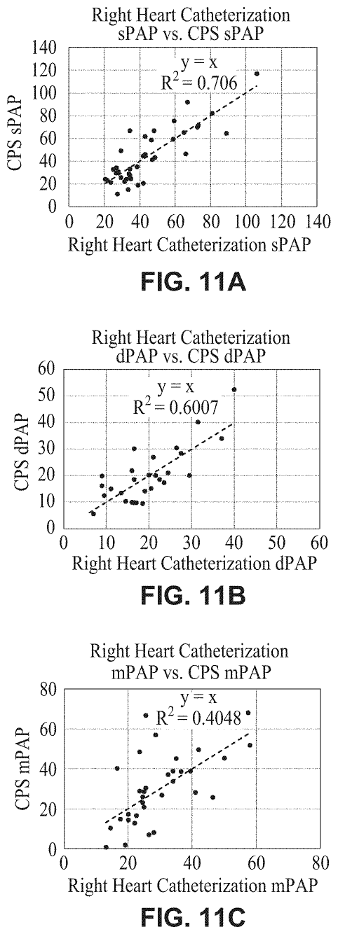

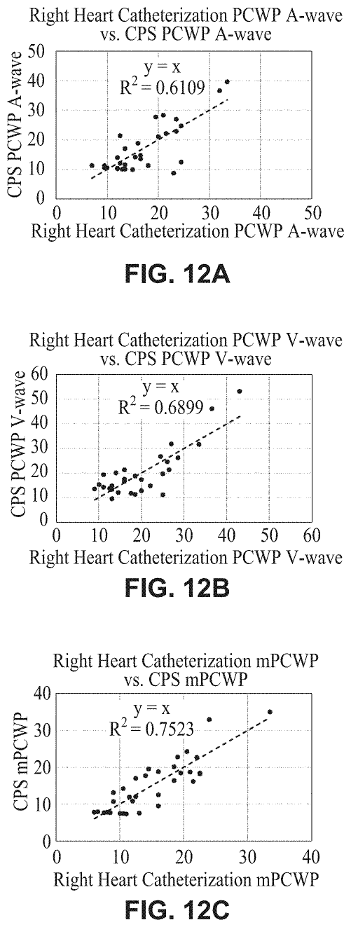

Furthermore, techniques of feature extraction at block 14 allow for the identification of feature value trends within a cardiac cycle, differences in feature values and/or feature value trends for PCG signals acquired across different sensor locations, and/or variations in feature values across the breathing cycle and may be used along with several well-known classification or regression techniques to compute PAP (and its components systolic-PAP, diastolic-PAP, mean-PAP) and PCWP (and its components PCWP A-wave, PCWP V-wave, and mean-PCWP). When the required steps of system training and calibration ensuring accurate measurement (prediction) of subject pulmonary pressure values are completed, the feature classifier or regression system is then configured with the calibrated classification or regression weights. The system is then capable of continuous operation without any further training or calibration to compute PAP and PCWP values. An example of the operation of a trained and calibrated system response is shown in FIGS. 11A, 11B, 11C, 12A, 12B, and 12C.

A number of features relating to temporal, amplitude-based, and spectral characteristics are extracted from PCG signals at block 14. Features correlating strongly with pulmonary pressures extracted at block 14 preferably capture: (1) the intra-heartbeat variations in heart function 16 (for example, as measured by differences in computed feature values between segments of the same heartbeat for one cardiac cycle); (2) the anatomical variations in left and right heart function 18 (for example, as measured by differences in computed feature values for PCG signals acquired across different sensor locations on patient left vs. patient right), and/or (3) variations in feature values across the breathing cycle 20 (for example, as measured by the detection of changes in heart sound characteristics with corresponding changes in lung air volume, intrapulmonary pressure, and/or intrapleural pressure during a breathing cycle).

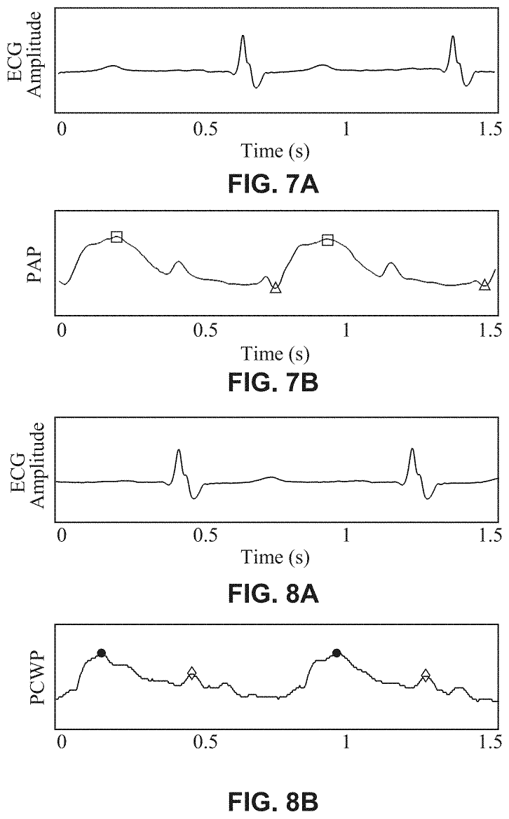

An example of a PAP waveform acquired during the right heart catheterization procedure is shown in FIG. 7A and FIG. 7B. Here, the ECG signal for two consecutive cardiac cycles is shown in FIG. 7A for comparison to a simultaneously acquired PAP waveform shown in FIG. 7B. The sPAP value is marked on the PAP waveform by squares and dPAP value is marked by triangles in FIG. 7B. The mPAP value is calculated as the average PAP value throughout each cardiac cycle.

Similarly, an example of the PCWP waveform acquired during the right heart catheterization procedure is shown in FIG. 8A and FIG. 8B. Here, the ECG signal for two consecutive cardiac cycles is shown in FIG. 8A against a simultaneously acquired PCWP waveform that is shown in FIG. 8B. The PCWP A-wave value is marked on the PCWP waveform by solid circles and PCWP V-wave value is marked by diamonds in FIG. 8B. The mPCWP value is calculated as the average PCWP value throughout each cardiac cycle. PCWP measurements are obtained at end-expiration to minimize the effect of the breathing cycle on intrathoracic pressures.

Another set of valuable features for extraction at block 14 are properties of formants in a PCG signal. These formants are concentrations of acoustic energy around a particular frequency in a PCG signal resulting from resonance of heart tissue, muscles, and blood during each cardiac cycle. For identifying these formants, the PCG signal belonging to the whole heartbeat or its segments may be first bandpass filtered with cutoff frequencies of 4 Hz and 100 Hz, for example. A compressed representation of the resulting signal can then be obtained using predictive modelling tools such as linear predictive coding. For this, the resulting signal may be first divided into smaller overlapping windows, for example, of a length of 32 samples with a 16 sample overlap between consecutive windows. The first n formants can then be extracted from the signal by computing the coefficients of the prediction polynomial returned by a linear predictive coding model of this signal of at least the (2n+2)th-order. The frequency and amplitude of the resulting formants as well as their trends and variations over time and location of signal acquisition can then be used to compute feature values that can track changes in intracardiac and pulmonary pressures throughout the cardiac cycle.

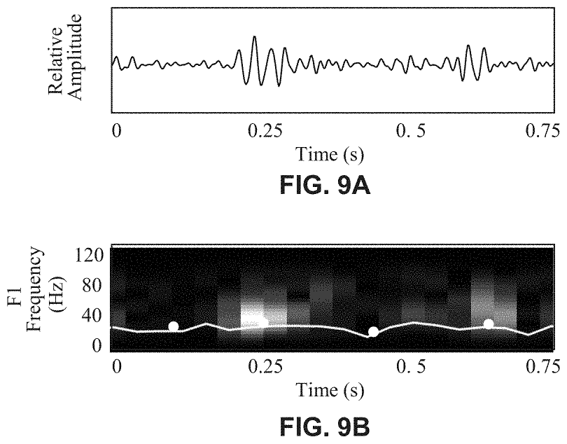

FIG. 9A through FIG. 9B illustrate an example of a formant frequency feature and its variations across different segments of the same heartbeat. FIG. 9A shows the bandpassed signal for the diastolic interval, S1, systolic interval, and S2 of a single heartbeat. FIG. 9B shows a plot of the frequencies of the first formant, F1, computed as described above overlayed on the spectrogram for this signal. Mean F1 frequencies for the diastolic, S1, systolic, and S2 segments are marked with solid circles. The instantaneous frequency of F1 and/or variations in the frequency of F1 across different segments may be used to characterize variations in intracardiac and pulmonary pressures throughout the cardiac cycle.

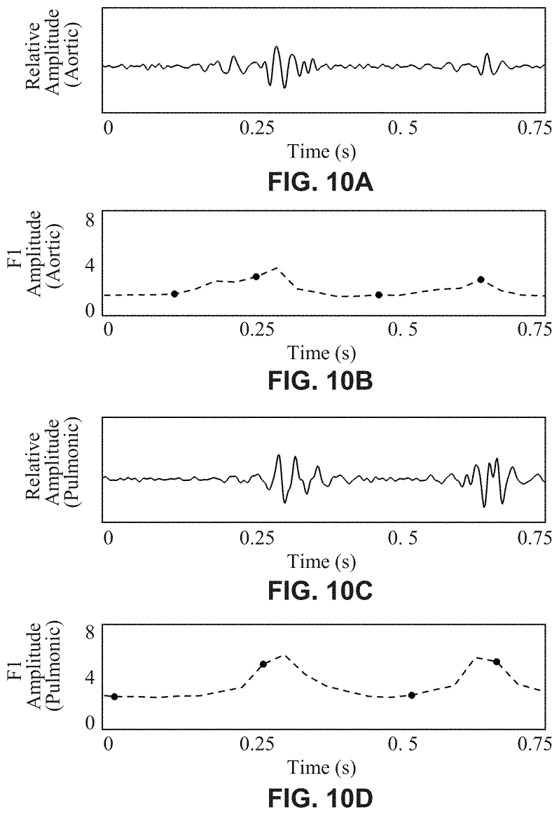

FIG. 10A through FIG. 10D illustrate an example of a formant amplitude feature for the same heartbeat as recorded at the aortic and pulmonic acoustic sensor locations. FIG. 10A and FIG. 10C show the bandpassed signal for the same heartbeat for the aortic and pulmonic site sensor locations, respectively. FIG. 10B and FIG. 10D show a plot of the amplitudes of the first formant, F1, computed in accordance with the present description for the signals from the aortic and pulmonic site locations, respectively. Representative F1 amplitudes for the diastolic, S1, systolic, and S2 segments are marked with solid circles. Comparisons of instantaneous or averaged F1 amplitudes across the two locations may be used to characterize variations in left and right heart function for the same cardiac cycle.

Other sets of features such as measures of central tendencies of the frequency distribution for a PCG signal or its segment, such as frequency center of mass or spectral centroid may also be used. Further, features characterizing the spectral entropy of a signal or its segment calculated as the negative product of the signal probability distribution for the selected PCG signal segment with its logarithm may allow for identification of signal segments with low values of spectral entropy and enable detection of coordinated heart muscle and tissue motion. Lastly, features that characterize breathing-related variations in heart rate and/or the shape of the PCG signal envelope obtained by applying a bandpass filter with example corner frequencies of 4 Hz and 100 Hz may allow tracking of PCG signal changes associated with the different phases of the breathing cycle.

Extracted features at block 14 are used as inputs to one or more previously trained and calibrated classification, regression, or advanced machine learning models at block 22 to produce pulmonary pressure (PAP or PCWP) values at block 24. Each component (systolic-PAP, diastolic-PAP, and mean-PAP for PAP, and PCWP A-wave, PCWP V-wave, and mean-PCWP for PCWP) has its own classifier and/or regression model at block 22 which is generated based on training data. This yields one final value per-subject for systolic-PAP, diastolic-PAP, and mean-PAP for PAP, and PCWP A-wave, PCWP V-wave, and mean-PCWP for PCWP at block 24.

FIG. 11A through FIG. 11C plot results of an illustrative PAP computation process for set of subjects. Computed sPAP, dPAP, and mPAP values are plotted against their corresponding PAP values measured by the right heart catheterization procedure. It can be seen that the PAP regression model accurately computes sPAP, dPAP, and mPAP values at block 24.

Similarly, FIG. 12A through FIG. 12C are plots of results of the PCWP computation process for an illustrative set of subjects. Computed PCWP A-wave, PCWP V-wave, and mPCWP values are plotted against their corresponding PCWP values measured by the right heart catheterization procedure in FIG. 12A, FIG. 12B, and FIG. 12C respectively. It is clearly seen that the PCWP regression model accurately computes PCWP A-wave, PCWP V-wave, and mPCWP values at block 24.

The methods of calculating and monitoring pulmonary pressures described herein are preferably implemented in a mobile, wearable sensing and computing apparatus with sensors such as that shown in FIG. 13. The system apparatus of CPS 100 illustrated in FIG. 13 enables both point-in-time and/or continuous PAP (and its components systolic-PAP, diastolic-PAP, mean-PAP), and PCWP (and its components PCWP A-wave, PCWP V-wave, and mean-PCWP) measurements with a wearable device that can provide clinicians with critical assessment metrics for cardiac care of an individual patient. Specifically, in one embodiment, CPS 100 utilizes a compact, wearable acoustic sensor devices and ECG sensor electrodes in a convenient patient belt or adhesive attachment application system. CPS performs signal processing computation to characterize heart sound signals that are generated by cardiac hemodynamic flow, cardiac valve, and tissue motion. Signal processing is accompanied with one or more classification, regression, or advanced machine learning methods to provide accurate computation of PAP and PCWP and their associated component values.

In one preferred embodiment illustrated in FIG. 13, CPS 100 generally employs an CPS patient monitor 102 coupled to acoustic and ECG sensors. The illustrated patient monitor 102 has a processor 104 with sensor inputs 106, memory 108, application software 110 and a display 112. The sensor input 106 of monitor 102 is operably coupled to CPS acoustic sensors 140 and ECG sensor electrodes 160 and receives signals from CPS acoustic sensors 140 and ECG sensor electrodes 160 via leads 154 or wirelessly.

Application programming 110 is provided within memory 108 for analyzing data from CPS acoustic sensors 140 and ECG sensor electrodes 160 via execution on processor 104. The programming and memory may also include long term data storage to provide a retrievable measurement history of the patient over time.

Patient monitor 102 may also comprise an interface display 112 for outputting computed analysis results. However, in an alternative embodiment, the computed results are transmitted to a display device such as a cellular telephone, touchscreen tablet device, or dedicated display monitor.

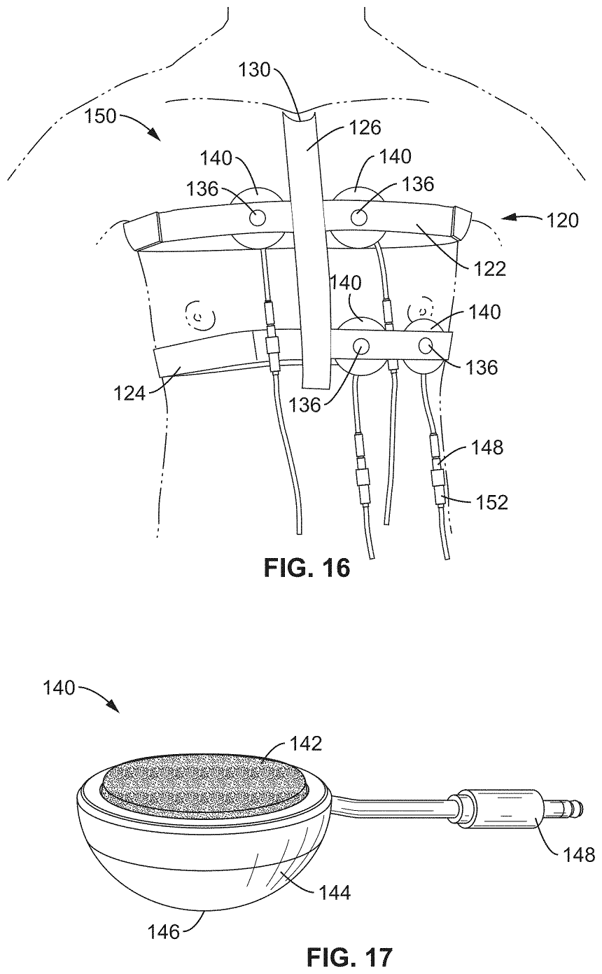

Although one CPS acoustic sensor 140 is shown in the embodiment of FIG. 13, multiple acoustic sensors 140 may be employed and positioned with CPS sensor support 120 to form an CPS sensor application system 150 as shown in FIG. 15 and FIG. 16.

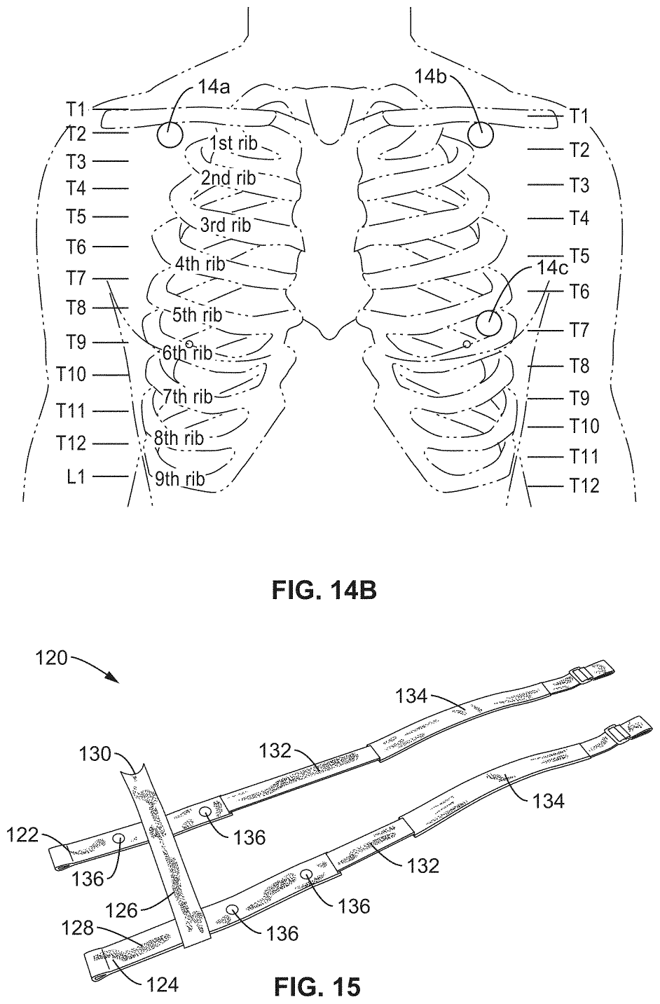

As will be explained in further detail below, an CPS sensor support 120 can be used that is configured to support CPS acoustic sensors 140 on the body of the patient at locations based on typical auscultatory sites like those identified in FIG. 14A as is used with a standard stethoscope system, e.g. aortic site location 12a, pulmonic site location 12b, tricuspid site location 12c and mitral site location 12d. In another embodiment, The CPS system 100 includes measurement capability for the CPS acoustic sensors 140 and standard three-lead ECG measurements. FIG. 14B shows representative ECG sensor electrode 160 locations 14a, 14b, and 14c applied at conventional RA, LA, and LL monitoring sites, respectively. In one embodiment, the CPS 100 system measures both acoustic signals from the four measurement sites 12a through 12d of FIG. 14A as well as the ECG signal from ECG sites 14a through 14c of FIG. 14B.

Computations of PAP and PCWP are based on analysis of S1, systolic interval, S2, and diastolic interval characteristics, their timing relative to the QRS event in the ECG signal, differences in them over time, differences in them as reflected across acoustic signals acquired at the multiple CPS acoustic sensor locations, and variations in them as reflected across the breathing cycle.

In an alternative embodiment, CPS is configured to monitor only acoustic signals from the CPS acoustic sensors 140 using a PCG-gated segmentation method, as provided in further detail above. In this system embodiment, ECG sensors, or other sensor input, are not necessary.

Positioning of sensors on the body of a patient at specific locations, such as shown in FIG. 14A and FIG. 14B, can be facilitated by a sensor support 120. In a preferred embodiment shown in FIG. 15 and FIG. 16, the CPS sensor support 120 of FIG. 15 is placed around the upper abdomen of a patient with characteristically positioned multiple CPS acoustic sensors 140 to form an CPS sensor application system 150. The support 120 of CPS sensor application system 150 holds CPS acoustic sensors 140 in position (e.g. at auscultatory locations 12a-12d) to allow for both point-in-time and/or continuous signal recording in a form that is comfortable for the patient, convenient and accurate for the care provider, and provides a low-cost disposable component enabling a single-use support.

FIG. 15 illustrates an embodiment of the CPS sensor support 120 with the acoustic sensors 140 removed for clarity. The CPS sensor support 120 includes two chest straps 122, 124 that are configured to be positioned horizontally around the patient as shown in FIG. 16. A vertical separator component 126 is fixed to the upper chest strap 122 and is configured to be attached via a releasable fastener 128 (e.g. hook-and-loop) to the lower chest strap 124. The vertical separator component 126 coupling the upper chest strap 122 and lower chest strap 124 indicates the vertical position of the two straps. A small semicircular indicator 130 at the upper end of the vertical separator 126 indicates the familiar and easily identified suprasternal notch of the sternum. The chest straps 122, 124 each include a pair of markers 136 that are configured to locate attachment of the CPS acoustic sensors 140 individually at preferred locations for acoustic monitoring within the abdomen/chest of the patient. Each of the horizontal chest straps 122, 124 preferably includes flexible stiffener sections 134 and elastic sections 132 for application convenience. All materials, including the elastic sections 132, are preferably composed of latex-free, biocompatible materials. In one embodiment, the CPS sensor support 120 is provided in a kit of varying sizes to match varying patient size, e.g. 5 sizes labeled X-Small, Small, Medium, Large, and X-Large.

FIG. 16 illustrates an embodiment of the CPS sensor support 120 with four acoustic sensors 140 to form an CPS sensor application system 150 positioned around the abdomen of the patient. With the semicircular indicator 130 at the upper end of the vertical separator 126 positioned at suprasternal notch of the sternum, the CPS acoustic sensors 140 are aligned at the proper locations for acoustic sensing, e.g. CPS acoustic sensors 140 on the upper chest strap 122 are aligned with the aortic site location 12a and pulmonic site location 12b, while the CPS acoustic sensors 140 on the lower chest strap 124 are aligned with tricuspid site location 12c and mitral site location 12d.

In one embodiment, the CPS sensor support 120 and/or CPS sensor application system 150 are configured as an adhesive-based disposable, single-use device ensuring proper and convenient attachment as well as patient comfort. In the embodiment shown in FIG. 13 and FIG. 16. four identical acoustic sensors 140 are shown applied to a subject. Each of the acoustic sensors 140 may have male 148/female 152 lead connections that are color coded for attachment to the CPS patient monitor via leads 154. FIG. 17 depicts a detailed, side perspective view of an illustrative CPS acoustic sensor 140 embodiment that can be used by CPS sensor application system 150. CPS acoustic sensor 140 comprises a half-dome shaped housing 144 with a nitrile (latex-free) membrane 142. At the opposite end 146 of the housing from the membrane 142, a releasable attachment means (e.g. circular area of hook-and-loop material-not shown) may be positioned to enable attachment of the acoustic sensor 140 to the CPS sensor support 120 at the specified markers 136. In one embodiment, the CPS sensors are configured as having adhesive stickers on top of the nitrile membrane that facilitates its adhesion to the subject's chest at the locations marked by the CPS sensor application system. It is appreciated that acoustic sensors 140, applied at each site, are connected to the patient monitor leads 152 with color-coded male connector 148 that matches the corresponding female connector 152.

This apparatus structure is an illustration of system structures that can be used in data acquisition and signal processing for computing PAP (and its components systolic-PAP, diastolic-PAP, mean-PAP), and PCWP (and its components PCWP A-wave, PCWP V-wave, and mean-PCWP) in accordance with the methods 10 of the present technology. The detailed methods are preferably implemented as instructions in machine-readable code within one or modules of application programing 110 of module 102, which may be executed and displayed on monitor 112 or other external processing device.

Accordingly, the CPS 100 can provide clinical patient care via convenient point-in-time and/or continuous monitoring ensuring patient safety with benefits to patients and clinicians as well as hospital facilities that can advance fundamental care. The system can also be used for outpatient treatment by providing cardiac function monitoring to patients who otherwise would not receive assessment as well as in residential monitoring, providing remote heart function diagnostic capability enabling early intervention and advanced perioperative care delivery.

The technology described herein may be better understood with reference to the accompanying examples, which are intended for purposes of illustration only and should not be construed as in any sense limiting the scope of the technology described herein as defined in the claims appended hereto.

### Example 1

In order to demonstrate the computation process for measuring PAP and PCWP values in a subject using the described methods, pulmonary pressure regression models were developed and trained from a set of subjects with available corresponding catheter-based measurements. The subject population that was chosen for developing the CPS pulmonary pressure regression models consisted of adult in-hospital patients undergoing an invasive right heart catheterization procedure. The selected subjects showed one or more cardiopulmonary afflictions such as congestive heart failure or pulmonary hypertension, etc.

During each catheterization procedure, a physician guided a special catheter into the pulmonary vessels of a subject's heart to observe blood flow and measure pulmonary pressures (sPAP, dPAP, mPAP, PCWP A-wave, PCWP V-wave, and mPCWP) as indicators of their heart and lung function. These catheter-based pulmonary pressure values were recorded as the ground truth for each subject. A CPS measurement was performed on each subject at the same time as the catheterization measurement. The acquired PCG acoustic and ECG signals were stored locally on the CPS patient monitor device. The data acquisition process was marked complete when CPS measurements and their corresponding catheter-based measurements were available for the entire set of subjects.

Later, the acquired signals were processed using MATLAB software to identify individual heartbeats and their segments, and signal features were extracted from these heartbeats. Individual per-heartbeat feature values were then averaged to obtain one overall feature value per subject. Multiple temporal, amplitude-based, and spectral features together constituted the CPS feature set. The best features among this set were those that showed strong linear relationships with the catheterization-based ground truth pulmonary pressure values across the entire set of subjects. Best features were selected for each of the six pulmonary pressures.

Thereafter, the selected top several best features for each pulmonary pressure value were used to train a regression-based neural network classifier to compute their corresponding CPS-based pulmonary pressure values. Each neural network consisted of an input layer, one or more hidden layers (each with one or several nodes), and an output layer. In this training process, each neural network learned the relationship between the input features and their corresponding ground truth pressure values over multiple iterations. In each iteration, the neural network produced an estimate for the CPS-based pulmonary pressure value as an output, evaluated this output value against the ground truth pressure value, and then sought to accordingly adjust the parameters of its next iteration. At the end of the training process, a successfully trained neural network was able to generate CPS-based pulmonary pressure values that were close approximations of catheterization-based pulmonary pressure values.

The success of the chosen features for predicting CPS-based pulmonary pressure values was determined using a leave-one-out cross-validation approach. Here, a neural network for a particular pressure value was first trained using features and ground truth values for all but one subject from the subject set. Next, this neural network was switched over from a learning operation to a running operation. In this, the feature value for the subject initially left out was provided as an input to the trained neural network to compute an CPS-based pressure value without knowledge of this subject's ground truth catheterization-based pressure value. This process was then repeated across the entire subject set, yielding a set of CPS-based pressure values for the entire subject set. The results of this validation process were visualized as plots of CPS-based versus catheterization-based pulmonary pressure values as seen in FIG. 11A through FIG. 11C. Each point on the plot represents a subject from the training dataset. The CPS-based pressure values as obtained from the leave-on-out cross-validation approach are shown on the y-axis, and the ground truth catheterization-based pressure values are shown on the x-axis. These plots validated the CPS pulmonary pressure regression models. Once trained and validated, static versions of these models were then available to be implemented for independent operation on new and never-seen-before subjects to compute PAP and PCWP values.

### Example 2

To further demonstrate the accuracy of the measurements of PAP or PCWP, the trained and validated CPS pulmonary regression models were used to obtain CPS-based pulmonary pressure values for new and never-seen-before subjects and compared to right heart catheterization results for the same subject.

For this, the trained and validated neural network model software was saved and transferred to a microprocessor in an CPS Patient Monitor as shown schematically in FIG. 13. Sensors were placed on the subject as illustrated in FIG. 14A and FIG. 14B. When an CPS measurement was performed on a new subject, the microprocessor performs steps of data acquisition, data processing, feature computation, providing features as input to the stored neural network model, and computing an CPS-based pulmonary pressure value for the subject in real-time without the need for any invasive right heart catheterization procedures. The accuracy of the of PAP or PCWP readings were later compared to catheterization results for select subjects to further validate the results of the methods.

Embodiments of the present technology may be described herein with reference to flowchart illustrations of methods and systems according to embodiments of the technology, and/or procedures, algorithms, steps, operations, formulae, or other computational depictions, which may also be implemented as computer program products. In this regard, each block or step of a flowchart, and combinations of blocks (and/or steps) in a flowchart, as well as any procedure, algorithm, step, operation, formula, or computational depiction can be implemented by various means, such as hardware, firmware, and/or software including one or more computer program instructions embodied in computer-readable program code. As will be appreciated, any such computer program instructions may be executed by one or more computer processors, including without limitation a general purpose computer or special purpose computer, or other programmable processing apparatus to produce a machine, such that the computer program instructions which execute on the computer processor(s) or other programmable processing apparatus create means for implementing the function(s) specified.

Accordingly, blocks of the flowcharts, and procedures, algorithms, steps, operations, formulae, or computational depictions described herein support combinations of means for performing the specified function(s), combinations of steps for performing the specified function(s), and computer program instructions, such as embodied in computer-readable program code logic means, for performing the specified function(s). It will also be understood that each block of the flowchart illustrations, as well as any procedures, algorithms, steps, operations, formulae, or computational depictions and combinations thereof described herein, can be implemented by special purpose hardware-based computer systems which perform the specified function(s) or step(s), or combinations of special purpose hardware and computer-readable program code.

Furthermore, these computer program instructions, such as embodied in computer-readable program code, may also be stored in one or more computer-readable memory or memory devices that can direct a computer processor or other programmable processing apparatus to function in a particular manner, such that the instructions stored in the computer-readable memory or memory devices produce an article of manufacture including instruction means which implement the function specified in the block(s) of the flowchart(s). The computer program instructions may also be executed by a computer processor or other programmable processing apparatus to cause a series of operational steps to be performed on the computer processor or other programmable processing apparatus to produce a computer-implemented process such that the instructions which execute on the computer processor or other programmable processing apparatus provide steps for implementing the functions specified in the block(s) of the flowchart(s), procedure (s) algorithm(s), step(s), operation(s), formula(e), or computational depiction(s).

It will further be appreciated that the terms “programming” or “program executable” as used herein refer to one or more instructions that can be executed by one or more computer processors to perform one or more functions as described herein. The instructions can be embodied in software, in firmware, or in a combination of software and firmware. The instructions can be stored local to the device in non-transitory media, or can be stored remotely such as on a server, or all or a portion of the instructions can be stored locally and remotely. Instructions stored remotely can be downloaded (pushed) to the device by user initiation, or automatically based on one or more factors.

It will further be appreciated that as used herein, that the terms processor, hardware processor, computer processor, central processing unit (CPU), and computer are used synonymously to denote a device capable of executing the instructions and communicating with input/output interfaces and/or peripheral devices, and that the terms processor, hardware processor, computer processor, CPU, and computer are intended to encompass single or multiple devices, single core and multicore devices, and variations thereof.

From the description herein, it will be appreciated that the present disclosure encompasses multiple implementations of the technology which include, but are not limited to, the following:

A method for measuring pulmonary artery pressure components (PAP) or Pulmonary Capillary Wedge Pressure components (PWCP) within a subject, the method comprising: (a) receiving phonocardiogram (PCG) acoustic signals from a plurality of acoustic sensors positioned on the chest of a subject; (b) segmenting the PCG acoustic signals to locate one or more cardiac events in the PCG acoustic signal; (c) extracting one or more €4 temporal, amplitude-based, and spectral characteristics from the segmented PCG acoustic signal; (d) applying one or more classification, regression, or advanced machine learning methods to the extracted characteristics to train, calibrate, and compute PAP and PCWP metrics and their components of a subject; and (e) outputting the computed PAP and PCWP metrics and their components of the subject for display; (f) wherein the method is performed by a processor executing instructions stored on a non-transitory memory.

The method of any preceding or following implementation, wherein segmenting the PCG acoustic signal comprises: detecting heart sounds within the PCG acoustic signal; identifying the heart sounds based on predefined criteria; labeling heart sounds as S1 and S2 based on an interval between successive events; and decomposing the PCG signal into individual cardiac cycles.

The method of any preceding or following implementation, further comprising: synchronously acquiring electrocardiogram (ECG) signals with the PCG signals from the subject; identifying R wave onset from the ECG signals; decomposing acquired ECG signals and PCG signals into individual cardiac cycles to segment the PCG signals.

The method of any preceding or following implementation, wherein identification of R wave onset from the ECG signals comprising: band-pass filtering the ECG sensor signal; multiplying the filtered signal by its derivative; computing an envelope of the multiplied signal; identifying R waves in the computed envelope; identifying corresponding peaks in the filtered signal; and determining an R wave onset in the filtered signal.

The method of any preceding or following implementation, wherein the cardiac events in the segmented PCG signal comprise: S1, systolic interval, S2, and diastolic interval within individual cardiac cycles.

The method of any preceding or following implementation, further comprising: preprocessing the PCG acoustic signal using Short-Time Spectral Amplitude Log Minimum Mean Square Error (STSA-log-MMSE) noise suppression; and wherein timing of the cardiac cycle based the acquired R wave onset is used to determine regions of acoustic inactivity as an input to STSA-log-MMSE.

An apparatus for monitoring pulmonary artery pressure (PAP) and pulmonary capillary wedge pressure (PCWP) in a patient, the apparatus comprising: (a) a plurality of acoustic sensors configured to be positioned on the chest of a patient; (b) a processor coupled to the plurality of CPS acoustic sensors; and (c) a non-transitory memory storing instructions executable by the processor; (d) wherein the instructions, when executed by the processor, perform steps comprising: (i) receiving a phonocardiogram (PCG) acoustic signal from the plurality of CPS acoustic sensors; (ii) segmenting the PCG acoustic signal to locate one or more cardiac events in the PCG acoustic signal; (iii) extracting one or more of temporal, amplitude-based, and spectral characteristics from the PCG acoustic signal; (iv) computing the PAP and PCWP and their components of the subject based on the extracted characteristics; and (v) outputting the PAP and PCWP and their components of the patient.

The apparatus of any preceding or following implementation, wherein the instructions, when executed by the processor, perform steps further comprising: preprocessing the PCG acoustic signal using Short-Time Spectral Amplitude Log Minimum Mean Square Error (STSA-log-MMSE) noise suppression; and wherein timing of the cardiac cycle based the acquired R wave onset is used to determine regions of acoustic inactivity as an input to STSA-log-MMSE.

The apparatus of any preceding or following implementation, wherein segmenting the PCG acoustic signal comprises: detecting heart sounds within the PCG acoustic signal; identifying the heart sounds based on predefined criteria; labeling heart sounds as S1 and S2 based on an interval between successive events; and decomposing the PCG signal into individual cardiac cycles.

The apparatus of any preceding or following implementation, wherein the instructions, when executed by the processor, perform steps further comprising: synchronously acquiring electrocardiogram (ECG) signals with the PCG signals from the patient; identifying R wave onset from the ECG signals; decomposing acquired ECG signals and PCG signals into individual cardiac cycles to segment the PCG signals.

The apparatus of any preceding or following implementation, wherein identification of R wave onset from the ECG signals comprises: band-pass filtering the ECG sensor signal; multiplying the filtered signal by its derivative; computing an envelope of the multiplied signal; identifying R waves in the computed envelope; identifying corresponding peaks in the filtered signal; and determining an R wave onset in the filtered signal.

The apparatus of any preceding or following implementation: wherein the PCG signal is analyzed in an envelope segment containing two consecutive cardiac cycles; and wherein the extracted amplitude characteristics comprise one or more of: the root-mean-square (RMS) of the PCG signal envelope segment normalized by RMS of the PCG signal of the entire cardiac cycle; the peak amplitude of the PCG signal segment, normalized by variance of the PCG signal of the entire cardiac cycle; and the peak amplitude of envelope segment, normalized by the envelope mean value for the entire cardiac cycle.

The apparatus of any preceding or following implementation, wherein the extracting one or more of temporal, amplitude-based, and spectral characteristics from the segmented PCG acoustic signal comprises: (a) band-pass filtering the PCG sensor signals; (b) extracting formants from the filtered PCG signals; (c) measuring amplitude and frequency of extracted formants; and (d) computing feature values.

The apparatus of any preceding or following implementation, wherein the formants are extracted with linear predictive coding models.

A system for measuring pulmonary artery pressure (PAP) and pulmonary capillary wedge pressure (PCWP) in a subject, the system comprising: (a) one or more acoustic sensors configured to be positioned on the chest of a subject; (b) one or more electrocardiogram sensors configured to be positioned on the chest of a subject; (c) a processor coupled to the plurality of acoustic sensors and electrocardiogram sensors; and (d) a non-transitory memory storing instructions executable by the processor; (e) wherein the instructions, when executed by the processor, perform steps comprising: (i) receiving a phonocardiogram (PCG) acoustic signal from the plurality of the acoustic sensors; (ii) receiving a phonocardiogram (ECG) signal from the electrocardiogram sensors; (iii) segmenting the PCG acoustic signal to locate one or more cardiac events in the PCG acoustic signal; (iv) extracting one or more of temporal, amplitude-based, and spectral characteristics from the PCG acoustic signal; (v) computing the PAP and PCWP and their components of the subject based on the extracted characteristics; and (vi) outputting the PAP and PCWP and their components of the patient.

The system of any preceding or following implementation, further comprising a display for displaying the output PAP and PCWP and their components.

The system of any preceding or following implementation, wherein segmenting the PCG acoustic signal comprises: a PCG-gated segmentation or an ECG-gated segmentation.

The system of any preceding or following implementation, wherein the extracting one or more of temporal, amplitude-based, and spectral characteristics from the segmented PCG acoustic signal comprises: (a) band-pass filtering the PCG sensor signals; (b) extracting formants from the filtered PCG signals; (c) measuring amplitude and frequency of extracted formants; and (d) computing feature values.

The system of any preceding or following implementation, wherein the formants are extracted with linear predictive coding models.

The system of any preceding or following implementation, wherein the computing the PAP and PCWP and their components, comprises: providing a pre-trained model for at least one PAP or PCWP component, the pre-trained model selected from the group of models consisting of classification, regression, or advanced machine learning models; inputting the extracted characteristics of the subject into the selected pre-trained model; and outputting a PAP or PCWP component value.

As used herein, term “implementation” is intended to include, without limitation, implementations, examples, or other forms of practicing the technology described herein.

As used herein, the singular terms “a,” “an,” and “the” may include plural referents unless the context clearly dictates otherwise. Reference to an object in the singular is not intended to mean “one and only one” unless explicitly so stated, but rather “one or more.”

Phrasing constructs, such as “A, B and/or C”, within the present disclosure describe where either A, B, or C can be present, or any combination of items A, B and C. Phrasing constructs indicating, such as “at least one of” followed by listing a group of elements, indicates that at least one of these group elements is present, which includes any possible combination of the listed elements as applicable.

References in this disclosure referring to “an embodiment”, “at least one embodiment” or similar embodiment wording indicates that a particular feature, structure, or characteristic described in connection with a described embodiment is included in at least one embodiment of the present disclosure. Thus, these various embodiment phrases are not necessarily all referring to the same embodiment, or to a specific embodiment which differs from all the other embodiments being described. The embodiment phrasing should be construed to mean that the particular features, structures, or characteristics of a given embodiment may be combined in any suitable manner in one or more embodiments of the disclosed apparatus, system or method.

As used herein, the term “set” refers to a collection of one or more objects. Thus, for example, a set of objects can include a single object or multiple objects.

Relational terms such as first and second, top and bottom, and the like may be used solely to distinguish one entity or action from another entity or action without necessarily requiring or implying any actual such relationship or order between such entities or actions.

The terms “comprises,” “comprising,” “has”, “having,” “includes”, “including,” “contains”, “containing” or any other variation thereof, are intended to cover a non-exclusive inclusion, such that a process, method, article, or apparatus that comprises, has, includes, contains a list of elements does not include only those elements but may include other elements not expressly listed or inherent to such process, method, article, or apparatus. An element proceeded by “comprises . . . a”, “has . . . a”, “includes . . . a”, “contains . . . a” does not, without more constraints, preclude the existence of additional identical elements in the process, method, article, or apparatus that comprises, has, includes, contains the element.

As used herein, the terms “approximately”, “approximate”, “substantially”, “essentially”, and “about”, or any other version thereof, are used to describe and account for small variations. When used in conjunction with an event or circumstance, the terms can refer to instances in which the event or circumstance occurs precisely as well as instances in which the event or circumstance occurs to a close approximation. When used in conjunction with a numerical value, the terms can refer to a range of variation of less than or equal to ±10% of that numerical value, such as less than or equal to ±5%, less than or equal to ±4%, less than or equal to ±3%, less than or equal to ±2%, less than or equal to ±1%, less than or equal to ±0.5%, less than or equal to ±0.1%, or less than or equal to ±0.05%. For example, “substantially” aligned can refer to a range of angular variation of less than or equal to ±10°, such as less than or equal to ±5°, less than or equal to ±4°, less than or equal to ±3°, less than or equal to ±2°, less than or equal to ±1°, less than or equal to ±0.5°, less than or equal to ±0.1°, or less than or equal to ±0.05°.

Additionally, amounts, ratios, and other numerical values may sometimes be presented herein in a range format. It is to be understood that such range format is used for convenience and brevity and should be understood flexibly to include numerical values explicitly specified as limits of a range, but also to include all individual numerical values or sub-ranges encompassed within that range as if each numerical value and sub-range is explicitly specified. For example, a ratio in the range of about 1 to about 200 should be understood to include the explicitly recited limits of about 1 and about 200, but also to include individual ratios such as about 2, about 3, and about 4, and sub-ranges such as about 10 to about 50, about 20 to about 100, and so forth.

The term “coupled” as used herein is defined as connected, although not necessarily directly and not necessarily mechanically. A device or structure that is “configured” in a certain way is configured in at least that way, but may also be configured in ways that are not listed.

Benefits, advantages, solutions to problems, and any element(s) that may cause any benefit, advantage, or solution to occur or become more pronounced are not to be construed as a critical, required, or essential features or elements of the technology describes herein or any or all the claims.

In addition, in the foregoing disclosure various features may grouped together in various embodiments for the purpose of streamlining the disclosure. This method of disclosure is not to be interpreted as reflecting an intention that the claimed embodiments require more features than are expressly recited in each claim. Inventive subject matter can lie in less than all features of a single disclosed embodiment.

The abstract of the disclosure is provided to allow the reader to quickly ascertain the nature of the technical disclosure. It is submitted with the understanding that it will not be used to interpret or limit the scope or meaning of the claims.

It will be appreciated that the practice of some jurisdictions may require deletion of one or more portions of the disclosure after that application is filed. Accordingly the reader should consult the application as filed for the original content of the disclosure. Any deletion of content of the disclosure should not be construed as a disclaimer, forfeiture or dedication to the public of any subject matter of the application as originally filed.

The following claims are hereby incorporated into the disclosure, with each claim standing on its own as a separately claimed subject matter.

Although the description herein contains many details, these should not be construed as limiting the scope of the disclosure but as merely providing illustrations of some of the presently preferred embodiments. Therefore, it will be appreciated that the scope of the disclosure fully encompasses other embodiments which may become obvious to those skilled in the art.

All structural and functional equivalents to the elements of the disclosed embodiments that are known to those of ordinary skill in the art are expressly incorporated herein by reference and are intended to be encompassed by the present claims. Furthermore, no element, component, or method step in the present disclosure is intended to be dedicated to the public regardless of whether the element, component, or method step is explicitly recited in the claims. No claim element herein is to be construed as a “means plus function” element unless the element is expressly recited using the phrase “means for”. No claim element herein is to be construed as a “step plus function” element unless the element is expressly recited using the phrase “step for”.

1. A method for measuring pulmonary artery pressure (PAP) components and Pulmonary Capillary Wedge Pressure (PWCP) components within a subject, the method comprising:
(a) receiving phonocardiogram (PCG) acoustic signals from a plurality of acoustic sensors positioned on the chest of the subject;
(b) segmenting the PCG acoustic signals to locate one or more cardiac events in the PCG acoustic signal;
(c) extracting one or more formants from the segmented PCG acoustic signal;
(d) training a machine learning model with extracted PCG acoustic signal formants from one or more subjects;
(e) applying one or more machine learning models to the extracted characteristics to compute PAP and PCWP metrics and their components of the subject; and
(f) outputting the computed PAP and PCWP metrics and their components of the subject;
(g) wherein said method is performed by a processor executing instructions stored on a non-transitory memory.

2. The method of claim 1, wherein segmenting the PCG acoustic signal comprises:
detecting heart sounds within the PCG acoustic signal;
identifying the heart sounds based on predefined criteria;
labeling heart sounds as S1 and S2 based on an interval between successive events; and
decomposing the PCG signal into individual cardiac cycles.

3. The method of claim 1, further comprising:
synchronously acquiring electrocardiogram (ECG) signals with said PCG signals from said subject;
identifying R wave onset from said ECG signals; and
decomposing acquired ECG signals and PCG signals into individual cardiac cycles to segment said PCG signals.

4. The method of claim 3, wherein identification of R wave onset from said ECG signals comprising:
band-pass filtering the ECG sensor signal;
multiplying the filtered signal by its derivative;
computing an envelope of the multiplied signal;
identifying R waves in the computed envelope;
identifying corresponding peaks in the filtered signal; and
determining an R wave onset in the filtered signal.

5. The method of claim 1, wherein the cardiac events in the segmented PCG signal comprise: S1, systolic interval, S2, and diastolic interval within individual cardiac cycles.

6. The method of claim 1, further comprising:
preprocessing the PCG acoustic signal using Short-Time Spectral Amplitude Log Minimum Mean Square Error (STSA-log-MMSE) noise suppression; and
wherein timing of the cardiac cycle based the acquired R wave onset is used to determine regions of acoustic inactivity as an input to STSA-log-MMSE.

7. An apparatus for monitoring pulmonary artery pressure (PAP) and pulmonary capillary wedge pressure (PCWP) in a patient, the apparatus comprising:
(a) a plurality of acoustic sensors configured to be positioned on the chest of the patient;
(b) a processor coupled to the plurality of acoustic sensors; and
(c) a non-transitory memory storing instructions executable by the processor;
(d) wherein said instructions, when executed by the processor, perform steps comprising:
(i) receiving a phonocardiogram (PCG) acoustic signal from the plurality of acoustic sensors;
(ii) segmenting the PCG acoustic signal to locate one or more cardiac events in the PCG acoustic signal;
(iii) extracting one or more of formants from the PCG acoustic signal;
(iv) providing a trained model that has been trained and calibrated on extracted PCG acoustic signal formants from one or more subjects;
(v) computing the PAP and PCWP and their components of the patient based on the extracted formants and the trained model; and
(vi) outputting the PAP and PCWP and their components of the patient.

8. The apparatus of claim 7, wherein said instructions, when executed by the processor, perform steps further comprising:
preprocessing the PCG acoustic signal using Short-Time Spectral Amplitude Log Minimum Mean Square Error (STSA-log-MMSE) noise suppression; and
wherein timing of the cardiac cycle based the acquired R wave onset is used to determine regions of acoustic inactivity as an input to STSA-log-MMSE.

9. The apparatus of claim 7, wherein segmenting the PCG acoustic signal comprises:
detecting heart sounds within the PCG acoustic signal;
identifying the heart sounds based on predefined criteria;
labeling heart sounds as 51 and S2 based on an interval between successive events; and
decomposing the PCG signal into individual cardiac cycles.

10. The apparatus of claim 7, wherein said instructions, when executed by the processor, perform steps further comprising:
synchronously acquiring electrocardiogram (ECG) signals with said PCG signals from said patient;
identifying R wave onset from said ECG signals; and
decomposing acquired ECG signals and PCG signals into individual cardiac cycles to segment said PCG signals.

11. The apparatus of claim 10, wherein identification of R wave onset from said ECG signals comprises:
band-pass filtering the ECG sensor signal;
multiplying the filtered signal by its derivative;
computing an envelope of the multiplied signal;
identifying R waves in the computed envelope;
identifying corresponding peaks in the filtered signal; and
determining an R wave onset in the filtered signal.

12. The apparatus of claim 7:
wherein the PCG signal is analyzed in an envelope segment containing two consecutive cardiac cycles; and
wherein the extracted amplitude characteristics comprise one or more of: the root-mean-square (RMS) of the PCG signal envelope segment normalized by RMS of the PCG signal of the entire cardiac cycle; the peak amplitude of the PCG signal segment, normalized by variance of the PCG signal of the entire cardiac cycle; and the peak amplitude of envelope segment, normalized by the envelope mean value for the entire cardiac cycle.

13. The apparatus of claim 7, wherein said extracting one or more formants from the segmented PCG acoustic signal comprises:
(a) band-pass filtering the PCG sensor signals;
(b) extracting formants from the filtered PCG signals;
(c) measuring amplitude and frequency of extracted formants; and
(d) computing feature values.

14. The apparatus of claim 13, wherein said formants are extracted with linear predictive coding models.

15. A system for measuring pulmonary artery pressure (PAP) and pulmonary capillary wedge pressure (PCWP) in a subject, the system comprising:
(a) one or more acoustic sensors configured to be positioned on the chest of the subject;
(b) one or more electrocardiogram sensors configured to be positioned on the chest of the subject;
(b) a processor coupled to the one or more of acoustic sensors and electrocardiogram sensors; and
(c) a non-transitory memory storing instructions executable by the processor;
(d) wherein said instructions, when executed by the processor, perform steps comprising:
(i) receiving a phonocardiogram (PCG) acoustic signal from the plurality of said acoustic sensors;
(ii) segmenting the PCG acoustic signal to locate one or more cardiac events in the PCG acoustic signal;
(iii) extracting one or more of formants from the PCG acoustic signal;
(iv) providing a pre-trained model for at least one PAP or PCWP component, said pre-trained model selected from the group of models consisting of classification, regression, or advanced machine learning models;
(v) inputting the extracted characteristics of the subject into the pre-trained model;
(vi) computing the PAP and PCWP and their components of the subject based on the extracted formants; and
(vii) outputting the PAP and PCWP and their components of the subject.

16. The system of claim 15, further comprising a display for displaying the output PAP and PCWP and their components.

17. The system of claim 15, wherein said instructions, when executed by the processor, further perform steps comprising:
receiving an electrocardiogram (ECG) signal from said an electrocardiogram sensors; and
segmenting a PCG acoustic signal with a PCG-gated segmentation or an ECG-gated segmentation.

18. The system of claim 15, wherein said extracting one or more of formants from the segmented PCG acoustic signal comprises:
(a) band-pass filtering the PCG sensor signals;
(b) extracting formants from the filtered PCG signals;
(c) measuring amplitude and frequency of extracted formants; and
(d) computing feature values.

19. The system of claim 18, wherein said formants are extracted with linear predictive coding models.

20. (canceled)
